(19) United States
(12) Patent Application Publication
(10) Pub. No.: US 2010/0168527 A1
(43) Pub. Date: Jul. 1, 2010

(54) Multiuse, multipurpose, detachable, wrap-around, velcro secured seat belt attachment (EZE-FLAP)

(76) Inventors: Lawrence A. Zumo, Baltimore, MD (US); Kesselly Zumo, Baltimore, MD (US)

Correspondence Address: Lawrence A. Zumo, 2701 Presbury St., Baltimore, MD, 21216, US

(21) Appl. No.: 12/317,842

(22) Filed: Dec. 30, 2008

(51) Int. Cl. A61B 5/02

(52) U.S. Cl. 600/301

(57) ABSTRACT

One embodiment of a seat belt attachment (3) for providing, recording and wireless transmission of electrode (6,7,8,9) sensor autonomic physiologic data as well as distance vector locator (10) which communicates at both ends with flexible attachment fasteners (1, 2) to the seat belt. The outer covering of the attachment is made of a flexible material, Velcro secured and folded such that it can easily move slidably without fracturing, along its axes. In addition, an outer layer of cloth is added for ease of sweat stain absorption. The longitudinal axis of the base contains two through holes or portals (11, 12) for blood pressure cuff (14) attachment and wireless transmission of data respectively. Other embodiments are described and shown.

### BACKGROUND

1. Field

This invention generally relates to a seat belt attachment (3), multiuse, multipurpose, detachable, wrap around, having the means to prevent sweat staining of the driver's or passengers clothes especially during hot and humid weather when the seat belt is worn. This attachment can have electrodes embedded (6, 7, 8, 9, 10) to measure, record, display or transmit wireless vital physiological signs of temperature, respiration and heart rate. To this seat belt attachment, a portable blood pressure cuff (14) with LED display (16) of heart rate and blood pressure will be attached via attachment port (11). The significance of this blood pressure cuff so attached is to make and become a useful aid for blood pressure, the silent killer and the single most important risk factor for cerebrovascular accident also called stroke, check and recording routine, hence immensely impacting and reducing disability and death from stroke and its antecedent consequences.

2. Prior Art

During hot and humid weather, it has become increasingly common that with seat belts on, sweating underneath the (usually and easily dirt-stained ) seat belt can stain the collar and shirts of the seat belt user, ie sudomotor embarassment. This invariably leads to the decrease compliance with and use of the seat belt. This device will reverse that trend, increase the use of the seat belt and help in decrease the mortality and morbidity associated with non use of the seat belt during car crashes.

Previously, U.S. Pat. No. 6,897,788 to Khair et al (2005) shows a wireless system protocol for telemetry monitoring for physiological data from the wireless biosensor transceivers of a electrocardiogram data acquisition system but this seat belt attachment will be for autonomic physiologic data (blood pressure, heart rate-static or continuous, temperature, respiratory rate, attached to a seat belt. U.S. Pat. No. 4,716,906 to Ruff (1986) describes a blood pressure cuff particularly for use with ambulatory blood pressure products adapted to be positioned on a limb of a body but it does not include an LED display for heart rate and blood pressure recordings nor is it attachable via a port to a seat belt attachment. This improvement will help immensely with stroke risk factor surveillance as a car driver or passenger can have this important vital sign measured once or frequently and recorded/stored on the car electronic system wirelessly via a portal (12) which can then be transmitted encrypted or non-encrypted as desired or importantly can be monitored via a global positioning vehicle locator system operator during critical or distress situations. This can then enable law enforcement or EMS officers and/or first responders to triage response capabilities in case of the mass or multiple casualities, medical emergencies like cardiac arrest, heart attack, strokes, sudden loss of consciousness, seizures, sudden death, sudden blood volume loss, unexpected labor of a pregnant female, etc. During medical emergencies to car drivers or occupants, or remote system operators can alert the appropriate officials to dispatch appropriate help in a rapid time dependent fashion. In search and rescue missions or car chases or cars falling off for example a cliff into a ditch or water, once seat belt remains attached, the person (s) vital signs can be monitored continuously until help arrives and the person is extricated. During heavy snow and when people are trapped far away in their cars, wireless monitoring of their vitals can be done until help arrives.

There are however limitations and restricted use of the Onstar Vehicle locator system that this proposed invention will very significantly improve upon. U.S. Pat. No. 4,891,650(1990) to Eliezer Sheffer requires the vehicle electrical system (including battery) to be operational. This is an obvious draw back in case of eg. a missing vehicle drawned out batteries. Additionally, the present vehicle location system is very costly and hence not economical. Importantly, the vehicle data transferred does not take in account the vital physiological data of the individual occupants of the vehicle. This obviously severely limits the public health applicability and medical usefulness of the current vehicle locator and monitoring systems. With the current available system, simultaneous presence of a functioning wireless technology, GPS monitor and intact vehicle electrical system/battery are required before effective operation of the current system. This is cumbersome and not efficient from a public health point of view. The proposed invention will be able to store and/or transmit data from the sensors, etc with simultaneous requirement of all three component to be operational at one time.

In the case of a mass casualty like a biological or chemical attack and with the EMS system overwhelmed, people can be instructed to quickly enter all available cars and with this attachment, sudomotor and autonomic physiological data can be monitored remotely and wireless, giving useful information the first responders for triaging and rescue purposes as they head to the scene.

In events of people being trapped for days in the snow or far away, places if they have their seat belts on, vital autonomic physiologic data can be gleaned from them to ascertain whether they are still alive, dead or near dying.

The attachment shall be of sufficient length to cover the wear from the shirt collar to the waist diagonally. The embedded electrodes shall be placed in the following order, given that the human heart is on the left side, except in situ inversus when it is on the opposite side; electrode 6 for temperature, 7 for heart rate, 8 for respiration rate, 9 and 10 for spare functions. The reverse order on those in whom wearing the seat belts from right to left with particular emphasis to have heart rate electrode near the heart coverage area as much as possible. The heart electrode can measure instantaneous or continuous heart rate as desired. This will be useful in those conditions/risk factors that are sometimes very difficult to measure in the hospital setting or at least requiring prolonged monitoring like paroxysmal atrial fibrillation, lone atrial fibrillation, tachybradycardia syndrome, dysautonomias like fluctuating hyper- or hypotension associated with eg. Guillian Barre syndrome

The attachment shall be of material that will be sweat absorbent material but easily slidable and durable enough to hold the electrodes in place. Color of the material can be varied and of choice. The two ends of the attachment via fasteners/clip on strong but thin enough smoothly move slidably through seat belt holder without much hinderance. The portal 12 to be used to attach device that measure independently and transmit wirelessly the autonomic data. The heart rate on the blood pressure LED display and that on the seat belt electrode 7 to be compared for scientific synchrony and validity. The attachment to be folded as described in FIG. 6.

Blood pressure cuff with anchor/metal rod stabilizer as shown in FIG. 7. This can be stored in the side doors until needed or via a small hook can be perched to the seat belt holder for easy access and convenience.

A separate electrode sensor embedded in the seat belt attachment can be configured so that the distance and vector of the passenger(s) from a stationary emergency medical monitoring system can be easily calculated wirelessly so that quick and effective search and rescue team or emergency medical personnel can reach the passengers and driver in a timely manner which in turn can potentially reduce morbidity, mortality or infirmity of the car occupants.

In conclusion, insofar as I am aware, no wireless monitoring system for physiologic autonomic data recording and transmission is currently available nor currently attached to a portable blood pressure cuff. The usefulness and effectiveness of this device in emergency situations, military battlefield scenarios, mass casualties and routine monitoring of the single most important risk factor of cerebrovascular accident, ie. hypertension is truly evident as there is no currently available system that can do what this device can do.

### SUMMARY

In accordance with one embodiment, a seat belt attachment performing many functions including autonomic physiologic data measurement and transmission wirelessly, as well as mechanical functioning as a cushion to absorb sweat and debris especially during the warm and humid temperatures/climate which can potentially stain the collar and shirt of the passengers and driver who use the seat belts. This allows the user to be complaint with state and federal safety laws without having second thought about staining of the shirt collars or shirts.

Accordingly several advantages are to provide instantaneous or periodic measurement and recording of autonomic physiologic data, including blood pressure, heart rate, temperature, respiration rate, distance calculator probe. These advantages will be apparent when this device is used on a large scale and the epidemiologic benefits quantified. This, I believe, will have a significant positive effect, on eg. controlling the number risk factor of devastating cerebrovascular accident/stroke as well as very important economic benefits for the general population at large. Still further advantages will become apparent from a study of the following description and the accompanying drawings.

### DRAWINGS

FIG. 1 is a perspective view of the seat belt attachment to show the orientation and environment in which the attachment will be used showing both the driver and front seat driver only.

FIG. 2 is a perspective unfolded view of the attachment and how it is applied to the seat belt.

FIG. 3 is a perspective view when the attachment is secured to the seat belt.

FIG. 4 is a close up view of the seat belt attachment showing the Velcro inner with the positions of the different electrodes to measure heart rate, temperature, respiration rate, blood pressure data transfer, distance and vector locator as well as arrows showing how the attachment will be wrapped around the seat belt

FIG. 5 shows a partially folded view of the attachment showing the outer Velcro on one end and the inner Velcro lining on the other end.

FIG. 6 shows a closeup view of the attachment when it is secured and fastened around the seat belt.

FIG. 7 shows a close up perspective view of the folded blood pressure cuff with its LED unit display of heart rate and blood pressure recording and how it is attached to the seat belt attachment via a connecting portal.

FIG. 8 shows a closeup view of the inner of the blood pressure cuff with its Velcro surface.

FIG. 9 shows the outer view of the blood pressure cuff with LED display unit, connecting cord and outer Velcro attachment.

### DETAILED DESCRIPTION

### First Embodiment

FIG. 1 is a perspective view of the seat belt attachment 3 to show the orientation and environment in which the attachment to the seat belt will be used showing both the driver and front seat driver only. Fasteners 1, 2 will be used to secure the attachment to the seat belt to ensure the durability and reliability of the attachment. These fasteners will flexible and thin so that they can be able to easily pass thru the seat belt holder without hinderance. The fasteners are made out of malleable metallic clip so that they can be easily and securely attached but flexible enough to be moved when the seat belt is not being removed to be stored back to his holder like when the passengers are disembarking from the vehicle. The vehicle could be a small car, large truck, van, airplane, astronaut rocket, army vehicles or other similar vehicles used by humans as means of transport.

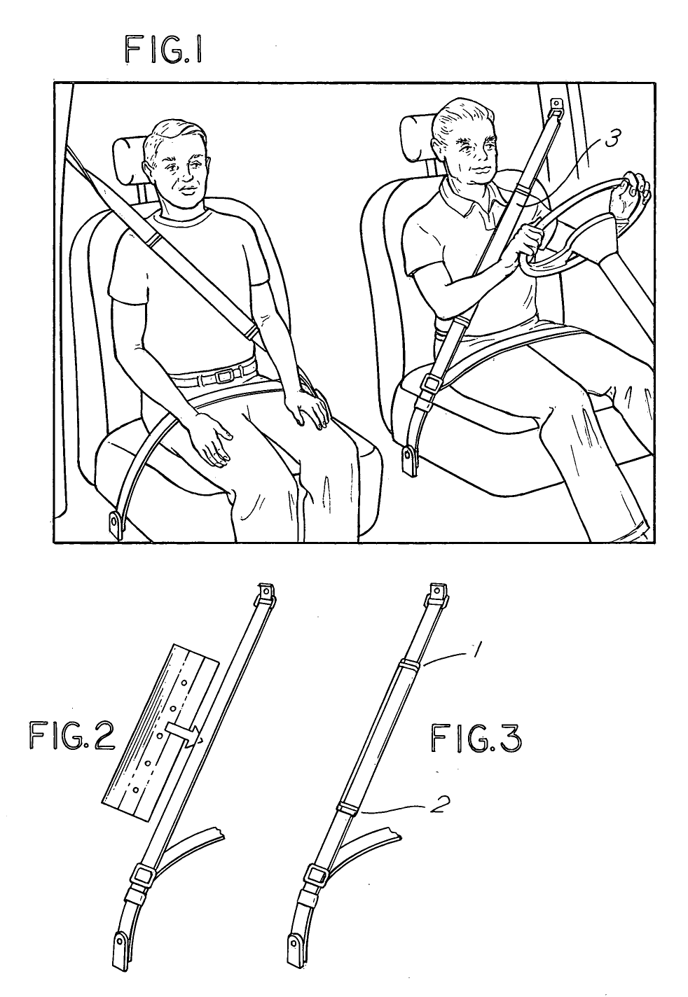

FIG. 2 is a perspective unfolded view of the attachment 3 and how it is applied to the seat belt, wrapping around and securing it appropriately so that when in use it will extend from the tip of the left shoulder to over the right ilium area to be attached to the fastener for those on left side of the vehicle including the driver and then vice versa attachment of the seat belt attachment following the original orientation of the seat belt on the right.

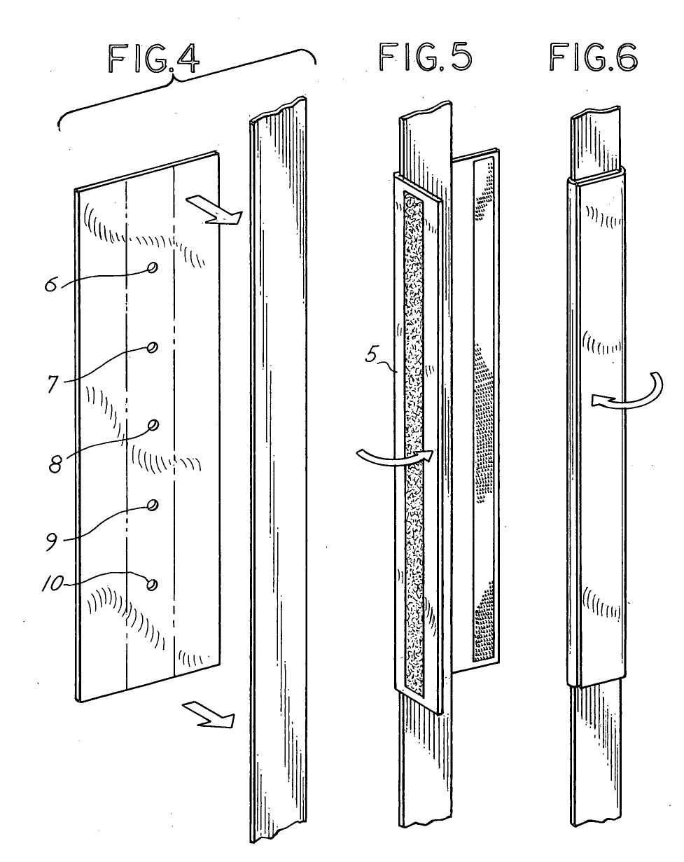

FIG. 3 is a perspective view when the attachment 3 is secured to the seat belt with malleable, flexible, thin fasteners 1, 2.

FIG. 4 is a close up view of the seat belt attachment 3showing the Velcro inner with the positions of the different electrodes to measure heart rate 7, temperature 9, respiration rate 8, blood pressure data transfer 6, distance and vector locator 10 as well as arrows showing how the attachment will be wrapped around the seat belt. These electrodes then will be configured to transmit these data to the car dashboard via portal 12 for easy viewing by the passengers and driver or transmitted wirelessly to a global positioning system or emergency medical or search and rescue command center for the most rapid response to save the maximum number of individuals in the shortest possible time in any and all medical emergencies. In the situation of the army, the distance and vector locator 10 can be disabled or encrypted to avoid falling in enemy hands.

FIG. 5 shows a partially folded view of the attachment showing the outer Velcro 4 on one end and the inner Velcro lining 5 on the other end which will make this attachment secure and useful.

FIG. 6 shows a closeup view of the attachment 3 when it is secured and fastened around the seat belt.

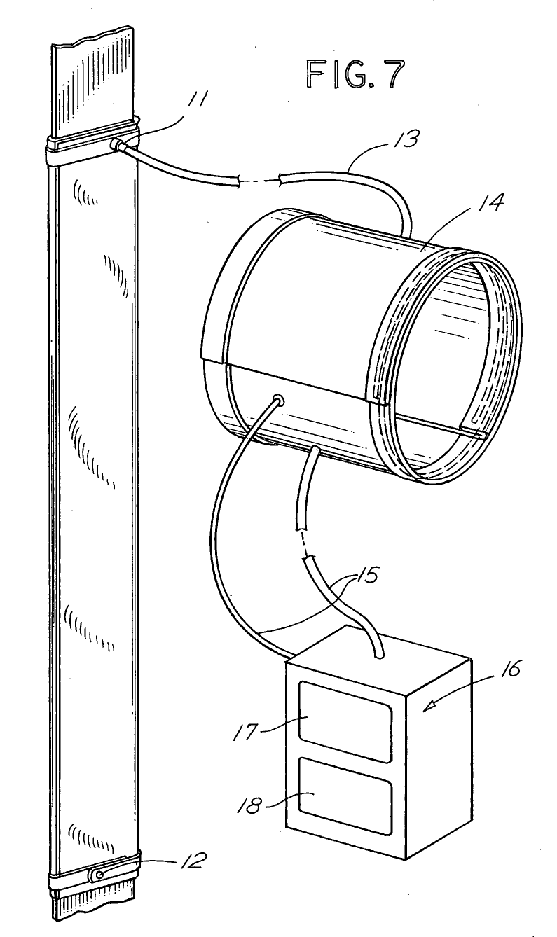

FIGS. 7,8,9—Additional Embodiments

FIG. 7 shows a close up perspective view of the folded blood pressure cuff 14 with its LED unit display 16 of heart rate 18 and blood pressure recording 17 and how it is attached 15 to the seat belt attachment 3 via a connecting portal 1 1 thru attachment cord 13. Portal 12 will be the site used for wireless device transfer of data. On the LED display there will be a button 25 which will serve as an on/off inflation/deflation button inorder for the blood pressure device to be turned on/off to inflate and record or deflate the blood pressure cuff 14. This will make it a lightweight efficient space saving, uninhindering seat belt attachment to measure and transmit vital autonomic data which should be used for the greatest medical good.

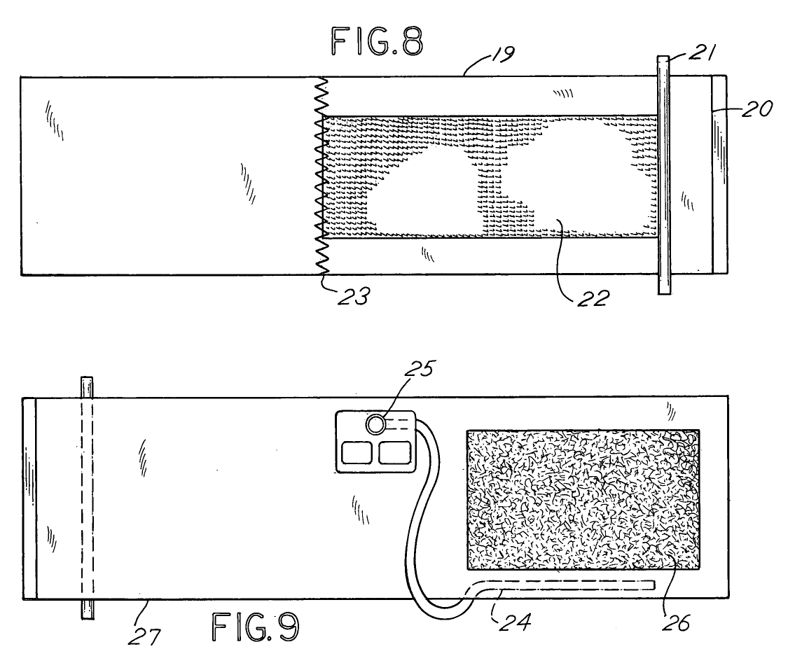

FIG. 8 shows a closeup view of the inner of the blood pressure cuff 14 with its inner Velcro surface 22. A stabilizing malleable plastic rod 21 will be so attached to the cuff so as to give the blood pressure cuff 14 anchor when it is being inflated or deflated.

The end of the blood pressure cuff will be doubly enforced so that the plastic rod 21 will remain secure during use. The inner rubber bladder of the cuff will be sewn with a cotton/linen material as shown at position 23 for durability and ease of use and care. The top portion of the cuff 19 will be thin but enforced to secure the top portion of the inner rubber bladder of the cuff.

FIG. 9 shows the outer view of the blood pressure cuff 14 with LED display unit 25 with its deflation/inflation on/off button, connecting cord 24 insertion and outer Velcro attachment 26 as well as the lower end of the blood pressure cuff thin but enforced cloth/linen end.

### Advantages

From the description above, a number of advantages of some embodiments of seat belt attachment become evident:
(a) The presence of this seat belt attachment will eliminate the unsightly staining of the occupants' shirts and apparel.
(b)The sensors will be able to record statically or in real time the very vital autonomic physiological data of each occupant of the car and can be transmitted in real time to emergency medical personnel as needed.
(c) The blood pressure attachment will provide an easy means to measure blood pressure of the occupants of the car, providing an important epidemiologic asset in monitor that very significant stroke risk factor.
(d) The advantage of this attachment over other wireless, GPS based systems is that it is cheap, tailored to individual automobile occupant and will not need the car battery or the fully functional car electrical system to become useful.
(e) It is simply attachable, flexible, multiple purpose-usable that will make it convenient, hence will ensure compliance with state mandated seat belt laws as well as provide important physiological data that will be of immense value to medical personnel and to emergency medical personnels as well.

### CONCLUSION, RAMIFICATIONS, AND SCOPE

Accordingly, the reader will see that, according to one embodiment of the invention, I have provided a seat belt attachment means to prevent sweat-staining of the car/motorized vehicle/aircraft occupant's clothes and apparel when secured as instructed. Futhermore, several vital physiologic data can be obtained and utilized fully from the public health and emergency medical system point of view.

Although the description above contains many specificities, these should not be construed as limiting the scope of the embodiments but as merely providing illustrations of some of the presently preferred embodiments. For example, the seat belt attachment can be used as a mechanical device, a sweat stain absorbent as well as embeddent for physiologic and wireless enabled electrodes which can be crucial from public health, epidemiology and emergency medical services point of view, etc. Thus the scope of the embodiments should be determined by the appended claims and their legal equivalents, rather than by the examples given.

### REFERENCE NUMERALS

1 top attachment fastener
2 lower end of attachment fastener
3 seat belt attachment
4 outer Velcro of attachment
5 inner Velcro of attachment
6 blood pressure electrode/sensor
7 heart rate sensor
8 respiration rate sensor
9 temperature sensor
10 distance and vector locator
11 blood pressure cuff attachment portal
12 wireless transmission portal
13 connection cord of blood pressure cuff
14 folded blood pressure cuff
15 connection cord between pressure cuff and LED display unit
16 LED display unit
17 blood pressure display on LED unit
18 heart rate display on LED unit
19 top end of blood pressure cuff
20 reinforced end of blood pressure cuff
21 malleable plexiglass rod as anchor on cuff
22 inner Velcro layer of blood pressure cuff
23 sewn layer of inner bladder and covering linen of cuff
24 connecting cord of LED unit to inner rubber bladder
25 LED unit with on/off deflation/inflation button
26 outer Velcro layer of blood pressure cuff
27 lower end of thin blood pressure cuff

### Operation

In operation, one uses the seat belt in a normal manner but with the seat belt attachment 3 wrapped around and fastened around the seat belt, the user whether driver or passenger of the vehicle can, when desired record or transmit via sensors 6, 7, 8, 9, 10 autonomic physiologic data and distance vector locator respectively (FIG. 4 and 7). When the seat belt attachment is fastened and secured around the seat belt with sensors fully functional, six effects increase the seat belt and its attachment utility:
(1) the covering of the seat belt attachment 3 even under very hot, humid weather,the human user's shirt collar and shirt/upper garment will not be soiled with embarrassing sweat tinged stains.
(2) Sensor 6 will record and/or transmit blood pressure values instantaneously or intermittent to vehicle dash board or to remote emergency medical or control and command center
(3) Sensor 7 will record, store and/or transmit wirelessly heart rate instantaneously or intermittent to dash board or remote site. This can be very useful in search and rescue mission, or eg. monitor of abnormal heart rhythms like paroxysmal atrial fibrillation or supraventricular tachycardia
(4) Sensor 8 will record and/or transmit respiratory rate to command and monitoring site
(5) Sensor 9 will record and/or transmit via a transceiver ambient or surface body temperature to command and monitoring site. This is particularly vital in search and rescue missions.
(6) Sensor 10 will be configured to give the vehicle's distance vector coordinate wireless to ease of finding vehicle during search and rescue mission or during emergency rescue medical situations. This sensor can be disabled or encrypted when used in a military setting to avoid compromising location of army troops during combat operations.
   When user wishes to disembark from the vehicle as is done routinely, it is only necessary to unbuckle the seat belt and the attachment 3 will move slidably smoothly thru the belt holder (FIG. 1) to be stored until further use. With these additional features, seat belt use will be more widespread without need of additional legislation and/or police enforcement-yield very positive economic savings and incentives.

1. A means for the measurement, recording and transmission of continuous or instantaneous individual vital human autonomic physiologic data, comprising:
a) an electrode sensor for the measurement of heart rate,
b) an electrode sensor for the measurement of respiratory rate,
c) an electrode for the measurement of human body temperature,
d) an electrode for distance and vector coordinates locator,
e) said means will have a connection portal at its uppermost elongated end for the attachment for a portal which will:
1. transfer independently measured heart rate, displayed on an attached LED display, and
2. transfer independently measured blood pressure, display on an attached LED display,after releasing or suppressing a blood of a human body by the inner wall of the apparatus after compressed air is introduced into the inner tube of blood pressure measuring cuff.

2. A seat belt attachment device according to claim 1, wherein said attachment has electrode sensors embedded in an orderly and clean, unhindered fashion to record, transfer, and transmit wirelessly, encryptedly or non encryptedly, vital individual human autonomic physiological data.

3. A seat belt attachment device according to claim 1, wherein said attachment will be of flexible cloth material of sufficient size, length and thickness, with fasteners are both ends moving slidably and smoothly to substantially accommodate use by a human being,
whereby said attachment provides an enlarged means for holding said electrode sensor means conveniently and protects said electrode sensor means from breakage and provides a means to prevent the sweat or debris staining of the garments of the human user during hot, humid or wet weather conditions.
